(19) United States
(12) Patent Application Publication
(10) Pub. No.: US 2017/0252194 A1
(43) Pub. Date: Sep. 7, 2017

(54) Procedure for Reducing Effective Stomach Volume

(71) Applicant: Giuseppe Tripodi, Middlebury, CT (US)

(72) Inventor: Giuseppe Tripodi, Middlebury, CT (US)

(21) Appl. No.: 15/057,429

(22) Filed: Mar. 1, 2016

(51) Int. Cl. A61F 5/00; A61B 6/00; A61B 6/12; A61B 34/20; A61B 1/273

(52) U.S. Cl. CPC A61F 5/0036; A61B 34/20; A61B 1/2736; A61B 6/12; A61B 6/481; A61F 5/003; A61F 5/0089

(57) ABSTRACT

A procedure for reducing the effective volume of the stomach of a patient involves a balloon module which is introduced into the stomach by either a gastroscope or through a catheter inserted through the stomach wall. The balloon module has a pair of positioning strings and a balloon with an integral protruding tube. After introduction into the stomach, the balloon is inflated and secured in position by the positioning strings. The position of the balloon in the stomach may be monitored by a contrast medium introduced into the balloon.

### BACKGROUND

This disclosure relates generally to gastric bypass procedures which are employed for reducing the functioning capacity of the stomach to thereby reduce the capacity of an individual to consume food with the objective of reducing or stabilizing the weight of the individual. More particularly, this disclosure relates to an alternative procedure to conventional gastric bypass procedures.

In conventional gastric bypass procedures or restrictive procedures, as typically illustrated in FIG. 1, an upper portion of the stomach is closed off—preferably by stapling. An end of the now bypassed jejunum portion is stapled or sutured at a distal end to the limb jejunum. An outlet is formed in a passage from the upper passage of the stomach to a proximal jejunal limb. The jejunal limb completes a bypass to the intestinal system. The route of the food, schematically illustrated in FIG. 1, traverses to the stomach via the jejunal limb to the intestinal system.

Conventional bypass surgery thus typically involves a significant invasion of the abdominal wall and a significant surgical modification of the stomach. The conventional surgery requires a precise surgical closing off of a portion of the stomach, or resectioning of the same, as well as closing a distal end of the bypassed jejunum portion and constructing an outlet from the stomach to a reconstructed passageway from the upper portion of the stomach to the intestine. The surgical invasion of the abdominal cavity provides a potential for infection. The surgical closing or resectioning of a portion of the stomach and a portion of the jejunum may result in unpredictable or even fatal complications even though a very skillful and proper surgical procedure has been successfully performed.

This disclosure is directed to an alternative procedure which significantly lessens the degree of invasion into the abdominal cavity, significantly lessens the amount of surgery and significantly lessens the potential for infection, morbidity, mortality and other complications. The disclosed procedure also provides a functionally effective procedure which may be efficiently reversed with no or minor adverse consequences.

### SUMMARY

Briefly stated, a procedure for reducing the effective stomach volume of a patient comprises providing a balloon attached to a port and at least two attached strings. The procedure further comprises insufflating the stomach and inserting the balloon and strings into the stomach. The procedure comprises inflating the balloon and employing the strings to position the balloon in the fundus location of the stomach and attaching the strings to the wall fascia. The procedure further comprises filling the balloon with a contrast medium, monitoring the balloon to determine a proper position and securing the balloon in position in the stomach.

The step of inserting the balloon and attached strings into the stomach further comprises passing it through a catheter into the stomach by a Seldinger technique. Alternatively, the step of inserting the balloon and the strings into the stomach further comprises using a gastroscope to pass the balloon and the strings into the stomach. The balloon is secured in position by securing the strings to the abdominal wall fascia of the patient. The step of monitoring the balloon is performed by an abdominal x-ray procedure and/or by direct vision through a gastroscope.

In one embodiment, a needle attached to a catheter is inserted into the stomach and the balloon and strings are passed through the catheter by a second Seldinger technique for introduction into the stomach. The balloon is filled with contrast medium by passing the contrast medium through an integral communication tube. Upon inflation, the balloon has a quasi-triangular or pear shape. The step of filling the balloon with a contrast medium preferably comprises injecting 500-700 ccs of Gastrografin™, Gastrin™ or barium fluid using a pressure pump. In another embodiment, the procedure further comprises removing the balloon by deflating the balloon through a catheter inserted into the stomach and disengaging the strings and removing the balloon through the catheter. In another embodiment, the procedure further comprises deflating the balloon through a catheter inserted into the stomach and removing the balloon through the mouth by means of a gastroscope.

A procedure for reducing the effective volume of the stomach of a patient comprises providing a balloon module comprising a balloon and attached positioner wherein the balloon has a deflated form and is inflatable to an inflated form. After insufflating the stomach, the balloon module is inserted in a deflated form into the stomach. The balloon is inflated to the inflated form. The positioner is employed to position the balloon in the fundus location of the stomach. The procedure comprises filling the balloon with a contrast medium and monitoring the balloon to determine a proper position. The balloon is secured in a stable position in the stomach by passing the positioner through the stomach wall and securing the positioner.

The step of inserting the balloon module into the stomach comprises passing the balloon module through a scope into the stomach or using a gastroscope to pass the balloon module through the esophagus into the stomach. The balloon is secured in position by securing the positioner to the patient. The step of monitoring the balloon is performed by an abdominal x-ray procedure. In an alternative embodiment, a needle attached to a catheter is inserted into the stomach and the balloon module is passed through the catheter for introduction into the stomach. The procedure further comprises removing the balloon by deflating the balloon, disengaging the positioner and removing the balloon from the stomach.

In one preferred embodiment, a balloon module for insertion into the stomach of a patient comprises a balloon having an integral communication tube defining a port and being composed of gastric resistant and latex inert material. The balloon is inflatable to have a generally triangular or pear shape dimensioned for reception in the stomach so as to allow food to bypass the balloon and exit the stomach. Two positioning strings are attached to the balloon and wrappable about the balloon when the balloon is in a deflated state.

### BRIEF DESCRIPTION OF THE DRAWINGS

FIG. 1 is a schematic anatomical view of a stomach and related anatomical structures illustrating a conventional gastric bypass surgery;

FIG. 2 is a schematic view of a balloon module in a deflated wrapped state and a partial view of a sheath through which the balloon module is passed into the stomach;

FIG. 3 is a schematic view of a balloon module with an inflated balloon in the stomach and further illustrating the shape of the balloon and its capability to be selectively positioned in the stomach;

FIG. 4 is a schematic anatomical view illustrating an alternative procedure in relation to the stomach, esophagus and duodenum which employs a balloon module and a catheter;

FIG. 5 is a schematic anatomical view illustrating a step in a procedure in relation to the esophagus, stomach and duodenum and further illustrating a balloon module and a gastroscope;

FIG. 6 schematically illustrates a step in the procedure further illustrating the stomach, the esophagus and the duodenum and a balloon and a catheter connecting the balloon;

FIG. 7 schematically illustrates an injection step in the procedure in relation to the esophagus, the stomach and the duodenum and further illustrating the injection of a contrast medium into the balloon using a pressure pump;

FIG. 8 schematically illustrates a stabilization step in the procedure in relation to the esophagus, the stomach and the duodenum wherein the balloon is stabilized by means of an integral communication tube and strings;

FIG. 9 schematically illustrates a securement step in the procedure in relation to the esophagus, the stomach and the duodenum and further illustrating the attachment of the balloon communication tube under the skin;

FIG. 10 is a schematic illustration of a deflation/inflation step in the procedure in relation to the esophagus, the stomach and the duodenum wherein the balloon may be deflated and inflated to control the size and the possible deflation due to rupture;

FIG. 11 is a schematic illustration of another positioning step in the procedure further illustrating in relation to the esophagus, the stomach and the duodenum and how the balloon may be positioned and the gastroscope subsequently removed;

FIG. 12 is a schematic illustration of a feature of the procedure in relation to the esophagus, the stomach and the duodenum and further illustrating that any rupture of the balloon would not harm the patient because the balloon is attached to the abdominal wall; and

FIG. 13 is a schematic illustration of a reversing step of the procedure in relation to the esophagus, the stomach and the duodenum wherein the ruptured balloon, schematically illustrated, may be withdrawn by cutting sutures and withdrawing the ruptured balloon through the mouth of the patient using an endoscope.

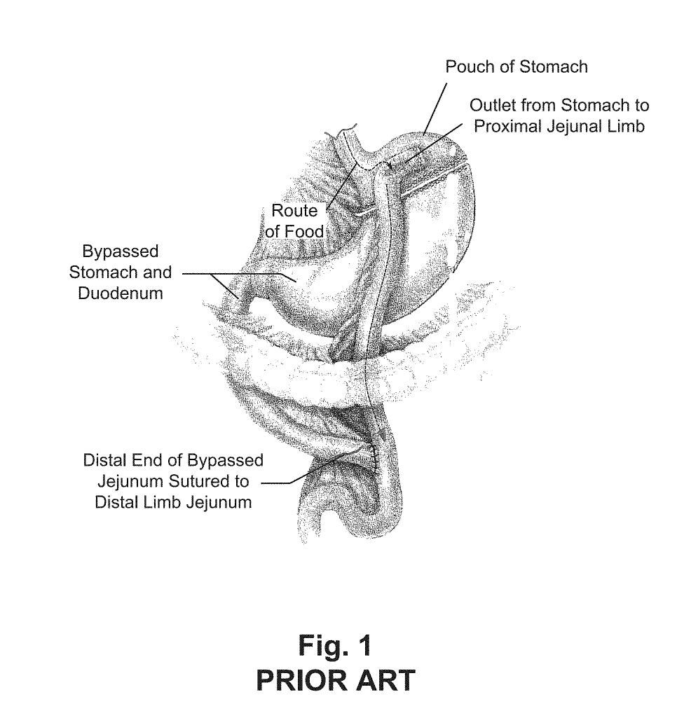

### DETAILED DESCRIPTION

With reference to the drawings which show various anatomical features of a representative patient, the anatomical features are designated by E for esophagus, S for stomach, F for fundus of stomach, D for duodenum and J for jejunum. It will be appreciated that the anatomical features of the drawings are intended to be representative and selective for purposes of illustrating the general features, and that naturally, there may be slight variations which will vary from patient to patient.

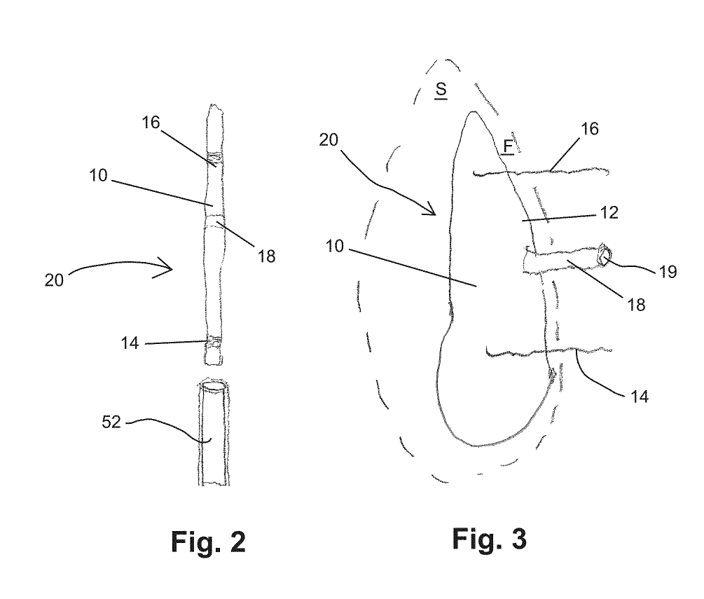

A key feature of the procedure for reducing the effective stomach volume is the usage of a specially configured balloon 10. The balloon 10 is initially in a deflated compact state (FIG. 2) and after insertion into the stomach, is inflated (FIG. 3).

With reference to FIG. 3, the balloon 10, upon inflation, assumes a generally triangular or pear-shaped configuration with an enlarged or bulbous portion 12 being adapted to seat against the fundus F of the stomach S. The balloon 10 may be somewhat distorted from the initial inflated shape when it is secured in position against a portion of the stomach. The balloon 10 is preferably manufactured from a material which is resistant to gastric fluids and preferably has a material composition which is inert to individuals who are allergic to latex. The balloon 10 has an extremely thin wall despite being resistant to rupture and has a high degree of structural integrity upon inflation.

At least two strings 14 and 16 are attached to the balloon to facilitate positioning and ultimate securing of the balloon in position. The strings are preferably composed of monofilament surgically compatible material such as employed for surgical stitches. The balloon 10 also includes an integral protruding fluid communication tube 18 having a distal port 19 which is openable and closable. The tube 18 functions for both positioning the balloon and for external fluid communication, as will be described below. The foregoing balloon 10, strings 14, 16 and tube 18 are designated as a balloon module 20.

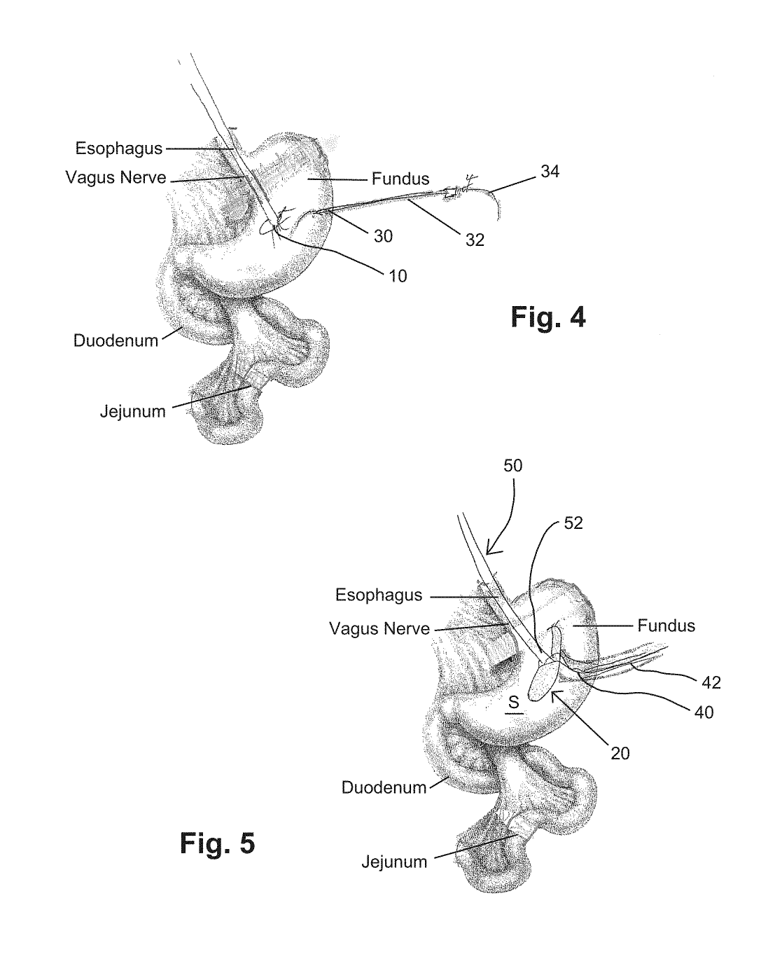

The balloon module 20 may be introduced in a fully deflated, compact wrapped form (FIG. 2) into the fundus of the stomach in one of two techniques. In a percutaneous method, or Seldinger technique, the insertion is made after insufflation of the stomach and under direct visual supervision by the surgeon. A trocar 30 connects with a sheath 32 and includes a guide wire 34 which introduces the balloon module 20 in a wrapped form with the attached strings into the stomach, as best illustrated in FIG. 4.

In an alternative technique best illustrated in FIG. 5, the balloon module 20 with the balloon tube 18 and the strings 14 and 16 are introduced into the stomach through a gastroscope 50. In this technique, the balloon 10, the tube 18 and the strings 14 and 16 are passed from the mouth to the stomach via an intragastric sheath 52 which will then be removed. Access to the tube 18 and the strings 14 and 16 is then realized through a percontaneous needle 40 with a suitable guide wire 42 for engaging the catheter and the strings. The needle 40 is inserted through a wall in the stomach S.

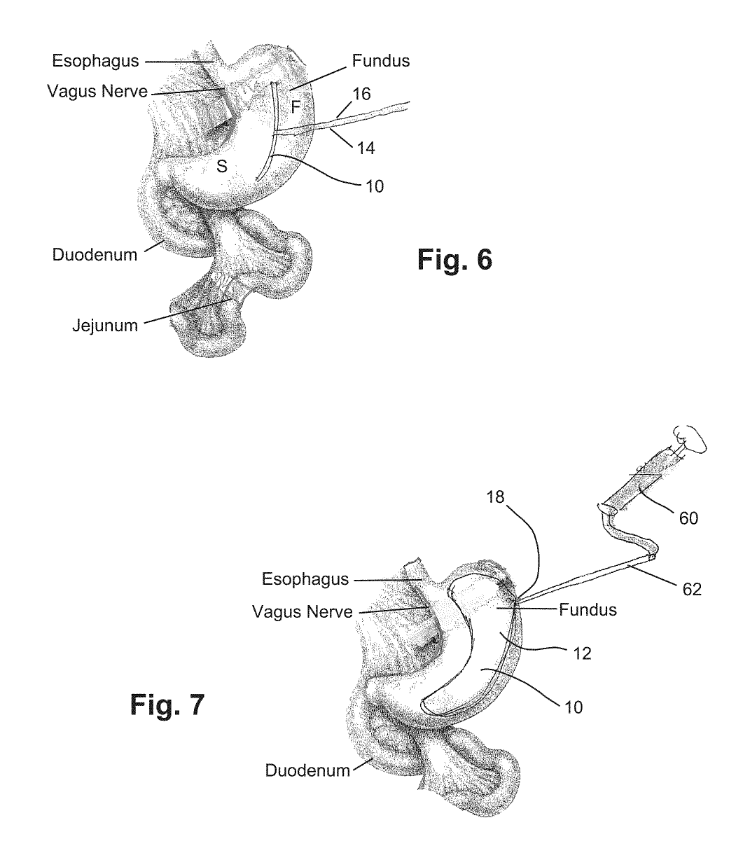

After removal of the introducer sheath 52, the balloon 10 is then positioned by the strings 14 and 16 and perhaps by the tube 18 so that the enlarged bulbous portion 12 of the balloon is drawn toward the fundus as illustrated in FIG. 6.

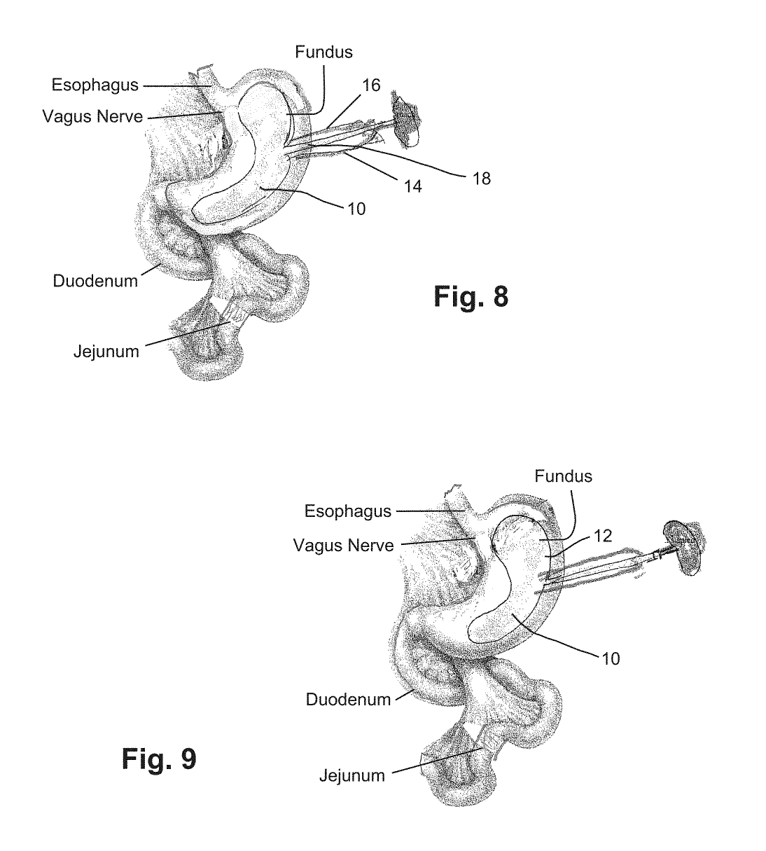

The next step is inflating the balloon. A pressure gauge 60 connects with an exterior conduit 62 which communicates with the tube 18. The balloon 10 is then filled with a contrast medium which is, in one embodiment, preferably 500 ccs-700 ccs of Gastrografin™, Gastrin™ or barium fluid, as best illustrated in FIG. 7. With reference to FIG. 8, the stabilization of the balloon is achieved by the tube 18 and by the two strings 14, 16 which are ultimately fixed to a pouch of tissue. The latter securement functions to impede dislodgement of the balloon which could potentially cause an obstruction.

After the inflation of the balloon and the attachment of the balloon, the port 19 is closed and the end of the tube 18 is secured in a pouch under the skin. The balloon can now be monitored. Preferably, this is accomplished by abdominal x-rays facilitated by the contrast medium to determine if any deflection or dislodgement or otherwise mispositioning of the balloon has occurred, as best illustrated in FIG. 9.

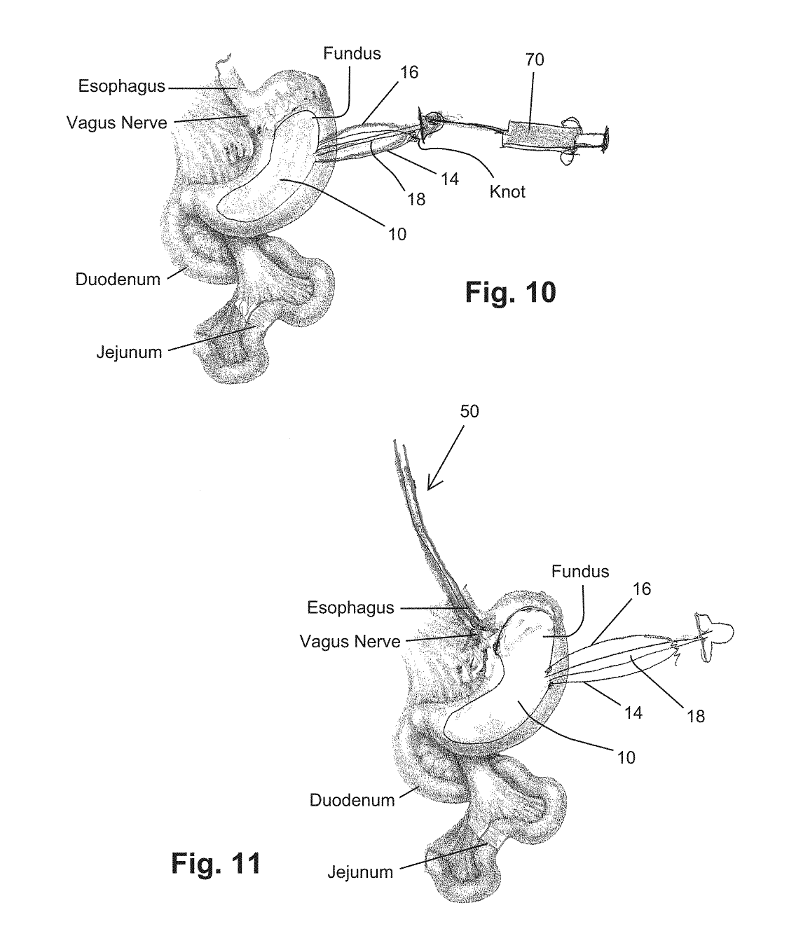

The balloon tube 18 is essentially closed once the proper positioning and configuration is achieved and secured. Any deflating or inflating of the balloon can be accomplished through a port of a pump 70 to control the size and possible deflation due to any rupture, as best illustrated in FIG. 10. Naturally, after proper positioning, the gastroscope will be removed and the conduit to access the balloon 10, the tube 18 and the strings 14, 16 will also be removed (See FIG. 11).

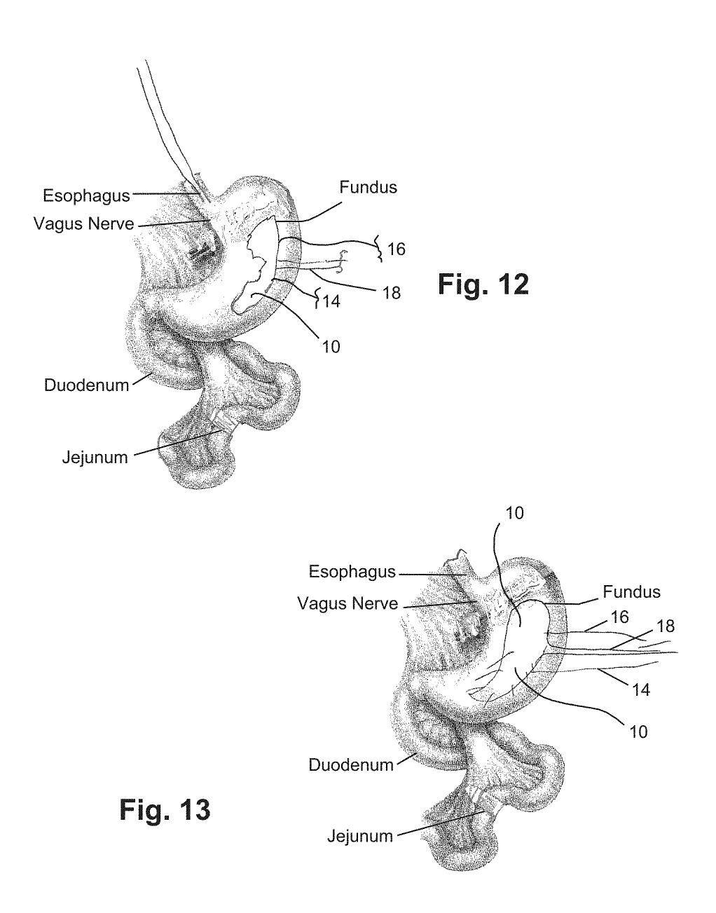

It will be appreciated that the rupture of the balloon for any cause would not cause any harm to the patient, such as intestinal obstruction, etc., because the balloon remains attached to the abdominal wall via the strings and the tube, as schematically illustrated in FIG. 12. Should a rupture of the balloon occur, the balloon can be easily removed. With reference to FIG. 13, the balloon tube 18 is detached from the pouch under the skin and the sutures are cut. An endoscope can then be inserted through the esophagus and engage the balloon to pull the balloon through the patient's mouth.

It will be appreciated that the procedure for reducing the effective stomach volume will eliminate any significant surgery to the patient and does not require a significant breaching of the abdomen or the stomach. In addition, the procedure can be relatively easily reversed should a breach of the wall of the balloon or a mispositioning of the balloon occur without significant harm to the patient and in a relatively efficient non-complex procedure.

While preferred embodiments of the foregoing have been set forth for purposes of illustration, the foregoing description should not be deemed a limitation of the invention herein. Accordingly, various modifications, adaptations and alternatives may occur to one skilled in the art without departing from the spirit and the scope of the present invention.

1. A procedure for reducing the effective volume of a stomach of a patient comprising:
providing a balloon having a port and at least two attached strings;
insufflating the stomach;
inserting the balloon and strings into the stomach;
inflating the balloon;
employing the strings to position the balloon in a fundus location of the stomach;
filling the balloon with a contrast medium;
monitoring the balloon to determine a proper position and inflation; and
securing the balloon in position in the stomach.

2. The procedure of claim 1 wherein the step of inserting the balloon and attached strings into the stomach further comprises passing the balloon and the strings through a catheter into the stomach.

3. The procedure of claim 1 wherein the step of inserting the balloon and the strings into the stomach further comprises using a gastroscope to pass the balloon and strings into the stomach.

4. The procedure of claim 1 further comprising securing the balloon in position by securing the strings to the patient.

5. The procedure of claim 1 wherein the step of monitoring the balloon is performed by an abdominal x-ray procedure.

6. The procedure of claim 1 further comprising inserting a needle attached to a catheter into the stomach and passing the balloon and strings through the catheter for introduction into the stomach.

7. The procedure of claim 1 wherein filling the balloon with a contrast medium further comprises passing the contrast medium through a catheter.

8. The procedure of claim 1 wherein said balloon further comprises a shape which, when inflated, has a quasi-triangular or pear shape.

9. The procedure of claim 7 wherein the step of filling the balloon with a contrast medium comprises injecting 500-700 ccs of Gastrografin™, Gastrin™ or barium fluid using a pressure pump.

10. The procedure of claim 1 further comprising removing the balloon by deflating the balloon through a catheter inserted into the stomach and disengaging the strings and removing the balloon through the catheter.

11. The procedure of claim 1 further comprising deflating the balloon through a catheter inserted into the stomach and removing the balloon through the mouth by means of a gastroscope.

12. A procedure for reducing the effective volume of a stomach of a patient comprising:
providing a balloon module comprising a balloon and an attached positioner wherein said balloon has a deflated form and is inflatable to an inflated form;
insufflating the stomach;
inserting the balloon module in a deflated form into the stomach;
inflating the balloon to the inflated form;
employing the positioner to position the balloon in a fundus location of the stomach;
filling the balloon with a contrast medium;
monitoring the balloon to determine a proper position; and
securing the balloon in a stable position in the stomach.

13. The procedure of claim 12 wherein the step of inserting the balloon module into the stomach further comprises passing the balloon module through a catheter into the stomach.

14. The procedure of claim 12 wherein the step of inserting the balloon module into the stomach further comprises using a gastroscope to pass the balloon module through an esophagus into the stomach.

15. The procedure of claim 12 further comprising securing the balloon in position by securing the positioner to an abdominal wall fascia of the patient.

16. The procedure of claim 12 wherein the step of monitoring the balloon is performed by an abdominal x-ray procedure or visual observation through a gastroscope.

17. The procedure of claim 12 further comprising inserting a needle attached to a catheter into the stomach and passing the balloon module through the catheter for introduction into the stomach.

18. The procedure of claim 12 wherein said balloon further comprises a shape which, when inflated, has a quasi-triangular or pear shape.

19. The procedure of claim 12 further comprising removing the balloon by deflating the balloon, disengaging the positioner and removing the balloon from the stomach.

20. A balloon module for insertion into the stomach of a patient comprising:
a balloon having an integral protruding tube defining a port and being composed of gastric resistant and latex inert material and being inflatable to have a generally triangular or pear shape which is dimensioned for reception in the stomach so as to allow food to bypass said balloon and exit said stomach; and
two positioning elements attached to said balloon and wrappable about said balloon when said balloon is in a deflated state.
